(19) United States
(12) Patent Application Publication
(10) Pub. No.: US 2011/0046673 A1
(43) Pub. Date: Feb. 24, 2011

(54) INTERSPINOUS DISTRACTION IMPLANT

(75) Inventor: MAURICIO RODOLFO CARRASCO, BUENOS AIRES (AR)

Correspondence Address: Mauricio Rodolfo Carrasco, Talcahuano 958 - 18th. Floor, Apt. C, Buenos Aires, C1013AAT, AR

(73) Assignee: PIXIS S.A, BUENOS AIRES (AR)

(21) Appl. No.: 12/772,904

(22) Filed: May 3, 2010

(30) Foreign Application Priority Data
May 4, 2009 (AR) P090101586

(51) Int. Cl. A61B 17/70

(52) U.S. Cl. 606/249

(57) ABSTRACT

This invention is directed to an interspinous distraction implant, which is intended to restrict the motion of the vertebrae between themselves, thus improving the mechanical behavior that is altered by the intervertebral disk diseases.

The invention is composed of an O-shaped body, which is oval and laminar, and includes two flaps for the implant anchorage and two additional pairs of upper and lower flaps wherein the upper flaps are forward with regard to the lower ones, so that both of them bump simultaneously into the respective vertebral laminae, thus avoiding the rotation of the implant in the transversal axis and positioning its longitudinal axis parallel to the spinous apophysis.

This invention is directed to an interspinous distraction implant, which is intended to restrict the motion of the vertebrae between themselves, thus improving the mechanical behaviour that is altered by the intervertebral disk diseases.

The invention is composed of an O-shaped body, which is oval and laminar, and includes two flaps for the implant anchorage and two additional pairs of upper and lower flaps wherein the upper flaps are forward with regard to the lower ones, so that both of them bump simultaneously into the respective vertebral laminae, thus avoiding the rotation of the implant in the transversal axis and positioning its longitudinal axis parallel to the spinous apophysis.

The invention is applied in the surgical field of the spinal area where it is necessary to keep the vertebrae separated, a relative motion between said vertebral bodies as well as the lumbar curve harmony and the spinal equilibrium.

### PRIOR ART

This invention pertains to the technology field related to those implants used in the spine surgery.

The interspinal distraction devices have been used for many years for the immobilization of the vertebrae aiming to achieve a definitive joining thereof, for instance in the case of an osteopathy. There exist several ways of making mountings in the vertebral bodies to achieve the immobilization of the vertebrae. A way of stabilizing the lumbar vertebrae is carried out by securing them by their top part, named neural arc, which is composed of laminae and spinous apophysis. The osteosynthesis systems that secure with bows to the laminae and/or spinous apophysis have been frequently used in the spinal surgery during the last eighty years. The objective these systems have in common consisted in immobilizing or restraining the mobility between the vertebrae in order to achieve a definitive joining.

As an example of the above given explanation, the use of bone as an element that is interposed between the spinous apophysis is described for the cervical area. These varieties of bows are profusely illustrated in the text “The Cervical Spine”, J.B. Lippincott Company and in the orthopedic surgery text book by Campbell, Chapter 56, Volume III.

In the lumbar area, the implants used to be placed between the spinous apophysis are built in different materials such as metal, plastic or elastomers. During the last twenty years, there has been an increase in the study of inventions intended to limit the motion of the spine without suppressing it completely. The surgical methods that are used to place said implants are known by the generic name “dynamic stabilization” or within the field of “Nonfusion Technologies”, “Nonfusion Technologies in Spine Surgery”, Szpalski and Col. Lippincott Williams & Wilkins 2007.

In general terms, the role of these implants when they are placed between the spinous apophysis, is to keep the vertebral bodies separated and limit the mobility between them in the three spatial planes. The mechanical expectation is to normalize the mobility that is altered by various diseases of the intervertebral disk which impair its holding function and which, secondarily, would alter the mobility between the vertebral bodies.

A variety of inventions that managed to become products may be analyzed in the work by Viscogliosi Bros, “Spine Non Fusion”, May 2004, wherein some of the most frequent devices used to this end are exemplified together with its action mechanics.

In said work, the device “Diam”, introduced by Medtronic, Sofamor Danek, is described. It is a device built in silicone and wrapped in a polyester mesh and two polyester cables, one of which protrudes from one side of the device and the other one, from the other side thereof. The surgical technique described by the same company considers the possibility of using or not using the polyester cables, so that their function consists only in keeping the implant in place to avoid the losing the position. Therefore, the use of these artificial links is an option and it should not be considered as an essential part of the technique. Its only action consists in separating the vertebrae by opening the rear part of the vertebrae and approaching the front vertebral edges. This type of implant does not limit the vertebral rotations. On the other hand, the surgical technique for its positioning requires the separation of the spinous apophysis by means of an instrument that lacks a strength meter so that the separation size achieved depends on each surgeon's strength. The surgical technique is carried out with the total exposure of the spinous apophysis and a wide separation of the aponeurosis towards both sides thereof.

With a similar conception, the product “Wallis” from Spine Next, U.S. Pat. No. 6,761,720, is an implant that, after having been positioned between the spinous apophysis, is held in place by means of two polyester bands which are afterwards passed by the neighbouring spinous apophysis and are tightly fastened by means of precise instruments. As described in the technique, the main function of this interspinous implant is to separate the spinous apophysis and limit the flexion and extension of the spinous apophysis by means of strong fixation bands that clasp the neighbouring spinous. Finally, the technique intended to the immobilize the vertebral bodies is only a stabilizing technique since the polyester bands that tightly clasp the spinous apophysis turn out to give way after some time thus enabling flexion and extension motions among the spinous apophysis as it may be seen in “Motion Preservation Surgery of the Spine”, James J. Yue. Saunders Elsevier 2008, page 528: FIGS. 69-14. This implant comprising a boy, a bow and several bow fastening elements, is finally composed of five pieces, it is not solid and it has two parallel cavities so that it is exclusively “softer”. This surgical technique requires the deinsertion of the supraspinous ligament and, afterwards, its suture. The invention is composed of a central body, two bows and two bow retention clips, which make a total of five pieces.

Another kind of implant is the “X-Stop”, which was introduced by St. Francis Medical Technology. It is a metallic cylinder in a more modern version, which is covered by a cylindrical elastomeric layer that is placed between the spinous apophysis separating them and is held in place by means of side metallic flaps, one of which protrudes from the same central metallic body, another one is added by means of a screw, which is fixed afterwards at the other end of the central metallic body and a last one is placed as a fastening accessory, which is a body comprising several pieces, a total amount of four pieces. The cylindrical elastomeric layer is solid and its elastic property is the one of its own composition and has no elasticity due to its shape that fits by pressing it into the metallic cylinder.

With special regard to the product “X-Stop”, it may be said that it separates the vertebrae but it does not enable that both vertebrae approach their apophysis. That is to say that it separates the vertebral bodies but it does not enable a natural motion of the spinous apophysis during the extension of the spine. The surgical technique is carried out with a total exposure of the spinous apophysis and a wide separation of the aponeurosis towards both sides of the spinous apophysis.

Another implant is the “Interspinous U Fixation System”, introduced by Fixano company. It is a metallic “U”-shaped device, U.S. Pat. No. 5,654,599, that separates the apophysis and said device is held by lateral protuberances being serrated on the sides that face the bones aiming to fix the implant and avoid its ejection. These flaps are fixed to the bones by various means such as cables or wires. This surgical technique requires the total resection of the interspinous ligament in order to make it possible to place the implant. Likewise, the thinning of the apophysis is necessary in order to place the flaps in their final position, as a consequence of which the bone is weakened. The surgical technique is performed with a total exposure of the spinous apophysis and a wide separation of the aponeurosis towards both sides of the apophysis. It is an implant composed of a body and two fastening elements to fasten the implant, joining the flaps to the bones of the spinous apophysis. Accordingly, the invention consists of three pieces.

Another negative aspect of the above-mentioned inventions is the awkward tying system to tie the ligaments or fastening cables, specially in the case of the “Wallis”, which requires a great exposure of the tissue and as a consequence of which there exists a considerable risk of infections. Moreover, it requires the withdrawal of the supraspinous ligament. Likewise, these ligaments make their biggest mechanical effort on the bone of the spinous apophysis, which may cause an osetolysis in the contact areas between the ligaments and the bone, thus weakening their mechanical action and promoting the system instability. Furthermore, these systems with a ligament that press the bones are not suitable for osteoporosis cases, in which fractures of the spinous apophysis have been reported due to minimum efforts thereon.

Therefore, there is the need of an interspinous distraction implant that reduces the surgical operation time and the exposure of the tissue during its insertion, be minimally invasive, limits the motion in the three spatial spaces in an elastic way, does not require bows, screws or other outer fastening elements that may compromise the spinous apophysis with additional forces.

### BRIEF DESCRIPTION OF THE DRAWINGS

FIG. 1 is front perspective view of the implant.

FIG. 2 is a rear perspective view of the implant.

FIG. 3 is a front view of the implant.

FIG. 4 is a rear view of the implant.

FIG. 5 is a front perspective view of the implant placed between two vertebrae.

FIG. 6 is a back perspective view of the implant placed between two vertebrae.

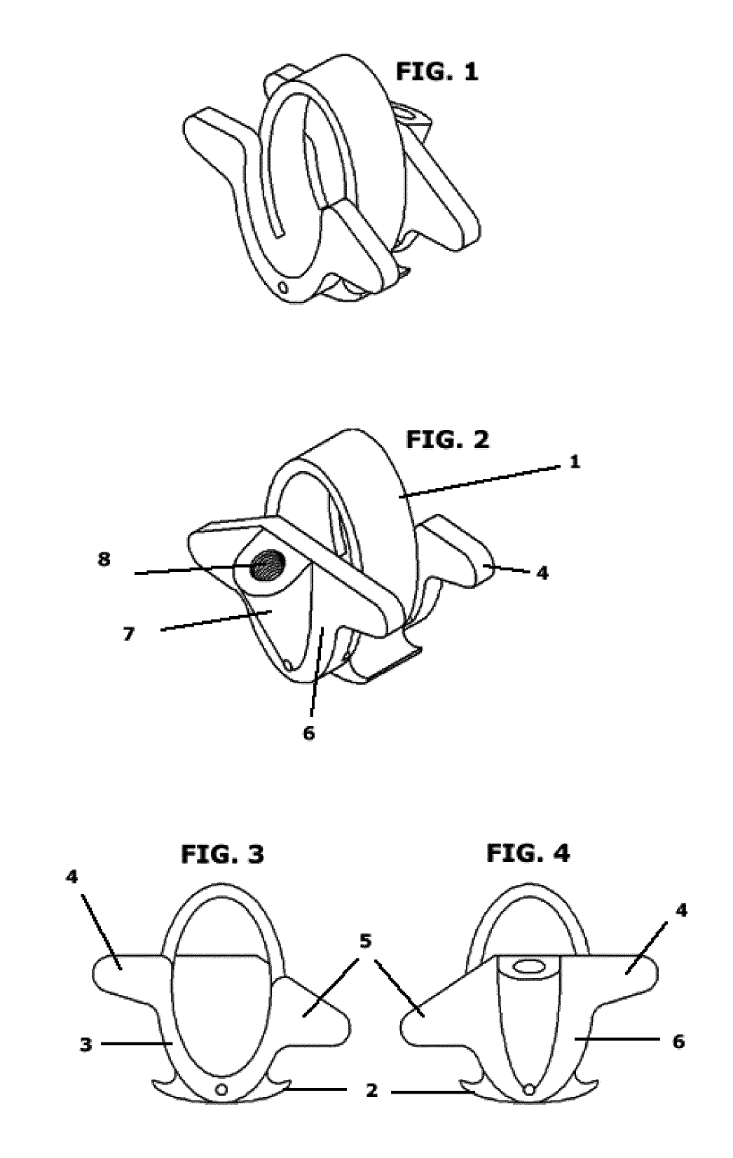

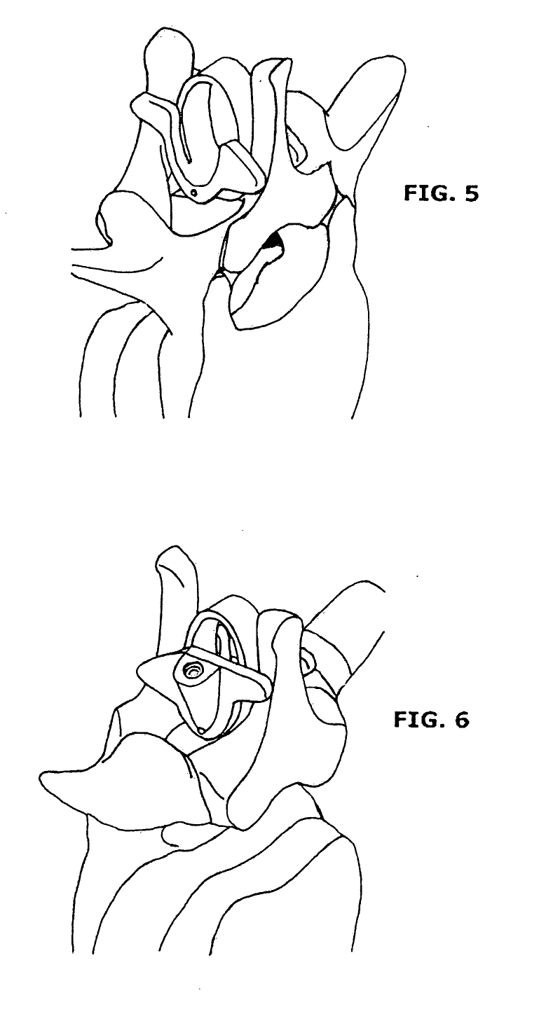

### PREFERRED EMBODIMENT

This invention consists in an interspinous distraction device, including an O-shaped (1), oval, laminar and asymmetric central body, being higher in its front part, and with an uneven thickness, the wider side of which is its front side and being reduced towards its rear part. At the bottom of the rear part, it includes two rectangular flaps (2) protruding towards the sides with ending edges for the anchorage of the implant and which prevent the motion thereof outside the interspinous space.

At the bottom, there is also a “U”-shaped extension towards the front part (3), corresponding with the shape of the central body (1) and in the ends of said U there protrude two asymmetric flaps, the upper one of which (4) is forward with regard to the lower one (5), so that both of them bump simultaneously into the respective vertebral laminae, thus avoiding the rotation of the implant around the transversal axis and positioning its longitudinal axis parallel to the spinous apophysis.

At said bottom, a backwards U-shaped extension is also included but said U shape is full (6), which corresponds with the shape of the central body and in the ends of said U there protrude two asymmetric flaps, the upper one of which (4) is forward with regard to the lower one (5), so that both of them bump simultaneously into the respective vertebral laminae, thus avoiding the rotation of the implant around the transversal axis and positioning its longitudinal axis parallel to the spinous apophysis. In the center of said full U, there is a protuberance included (7) which lodges a threaded hole (8) to hold a positioning surgical instrument. Said protuberance (7) is bent for illustrative purposes but it may also be parallel to the implant body or in the most comfortable position for the insertion of the implant.

There arises from the own anatomy of the vertebral laminae that the optimum position of the upper and lower flaps of this interspinous distraction implant will be that one wherein there is a minimum point in the lamina curvature or a widening that serves as a flap stop. From the study of said anatomy, there follows the need of advancing the upper flap or flap close to the head with regard to the lower flap or flap close to the feet so that both of them bump simultaneously into the area of the laminae that provide a more stable support.

As far as the O-shaped central body is concerned, a previous structural analysis was made in order to determine the ideal shape according to the burden of forces that the distraction implant should bear. This is the way in which an lying-U-shaped implant comprises the U bottom wherein practically all of the burden is concentrated. It requires an extremely resistant material, specially when flexibility is desired, such as is described in U.S. Pat. No. 5,645,599.

Unlike the U shape, the O-shape enables a more even distribution of the burden. On the other hand, the fact of having to hold the implant by means of screwed flaps creates an additional stress area, not only on the apophysis, which are already weakened by the holes that have been made to insert the screws, but also on the implant due to the burden the flaps receive during the flexion.

For this invention, there has also been considered an O shape that is the most suitable for the function of the implant subject to the burdens among the spinous apophysis. The O-shaped body that is in lying position has an end wider than the other one and a thinning in the upper and lower areas. The central body becomes deformed under the established burden. Under the determined burden, the following geometrical responses are established: the central body has a 8% flattening and a 3% stretch. This range corresponds to a central body with a height of 12 cm and 20 cm in length. This range may be adjusted.

A material suitable for the construction of the invention is the PEEK (Polyetiren-Etiren-ketone).

By virtue of the above-described embodiment and the material with which it has been built, said body elastically limits the mobility between the vertebral bodies in the three planes of the space and acts as a fulcrum only with its front part. Said central body has different thicknesses that determine different elasticity degrees during the compression of the vertebral bodies.

The central body has flanges on its outer surface with the possibility of acquiring a saw tooth configuration.

Said implant may have elements incorporated therein such as elements opaque to X rays, shaped or amorphous, and/or contain various substances having a biological action with antimicrobial agents, for the treatment of the metabolism, the skeleton and bone grafts. Likewise, it may carry electronic elements for measuring or therapeutic action.

Method for the Implant Positioning

After making a 4 mm incision on the spinous apophysis, the soft tissue is separated leaving the aponeurosis exposed. The aponeurosis is incised in only one of the sides of the spinous apophysis and the muscles of that side are separated. The ligament between the spinous apophysis is completely resected. The supraspinous ligament is separated towards the opposite side, also separating the muscle mass and the aponeurosis of the opposite side. At this step, while keeping the interspinous ligament separated from its insertion in the ends of the spinous apophysis, a measuring instrument out of a set of measuring instruments of different sizes is placed to measure the separation between the spinous apophysis and prepare the implant lodging. Once the size of the implant to be inserted had been selected from a set of implants of different sizes, the implant is mounted on an instrument to hold it by its lateral part and place it with said instrument into the space between the apophysis. The tissue is sutured.

The particular shape of the invention requires a particular and specific implantation technique, different from the one used for other inventions: the minimum surgical approach may be made with a particular and unique method. The way of preserving the supraspinous ligament in its original insertion is essential, as the way of keeping it separated protecting the aponeurosis and muscle mass of the side opposite the implant placing. If the supraspinous ligament were not preserved in its original insertion, the implant would remain unstable and there would be the possibility of its ejection.

The way of keeping it separated and protecting the aponeurosis and muscle mass of the side opposite the implant placing is essential and different from the techniques of the other implants, in which the muscle mass must be separated from both sides. This minor exposure makes a method of a minimum surgical invasion.

This characteristic is due to the fact that it does not require the resection of the supraspinous ligament and its surgical approach is limited, thus also limiting the tissue exposure to a 50% with regard to the exposure required in the case of other inventions, as a consequence of which the placing time is also reduced a 50%. Furthermore, in view of the fact that it does not require fastening elements such as polyester bows, wires or cables for its fixing, or the need to resort to another accessory fastening element, the insertion of strange material is also limited, thus avoiding a further element which may cause possible rejections of the organism or infections because of the need to make more surgical maneuvers for the positioning and more exposure time of the tissue to the outer environment.

This implantation technique reduces the tissue exposure, thus reducing bleeding, infection possibilities as well as the operation time. This surgical technique cannot be carried out with the other above-described inventions due to incongruities related to the implant shapes and the intended working.

Mechanical Performance and Working

Once the implant had been inserted between the spinous apophysis and the person is stood up, the implant acts a fulcrum in its front part in view of its bigger thickness and the fact of being less deformable, bearing the biggest compression effort between the vertebrae in that site and, in that way keeping the vertebrae separated in their front part. This aspect is essential for all of the implants that are intended to obtain a dynamic stabilization.

The novelty is based on the fact of having studied and achieved the construction of implants of different sizes to adapt them to different anatomies and that comply with the mechanical requirements of the lumbar area, specific of the spinous apophysis area, as well as having needed only one body without the need of accessories, suitable for the elastic support of the vertebrae, also that serve not only for the demands of the daily life but also in the case of accidental falls, which sharply raise the pressure between the vertebrae and strongly affect the spinous apophysis, fracturing them with an increased risk in the case of osteoporosis, and that avoid possible implant ejections or dislocations due to the lack of sufficient elasticity to adapt harmonically to said movements.

This elastic adaptation mechanism of the invention is based on the fact that when the spine is extended, the spinous apophysis flexes on the device thus causing a progressive pressure on the oval towards its thinner rear part, which is elastically deformed and which recovers its shape when the position of the spine is inverted. In this way, the invention acts as a spring for the flex-extension movement of the spine, with a pivot point in the front part of the implant, thus limiting the approach of the vertebral bodies, but without limiting the flexion and extension.

On the other hand, unlike the other implants that limit the rotation of the vertebral bodies by means of inelastic stops, the present invention has elastic laminar extensions from the central body, which elastically limit the rotation and, obviously, with more natural mechanics.

The above-mentioned extensions have a polymeric material thickness suitable to keep a certain elasticity degree. This elastic limitation is proper to limit the rotation of the vertebral bodies between each other in a precise degree. The alternative uses of the invention enable its use for the temporarily stabilization or as a carrier of biological substances, whether solid or liquid, in order to promote the definitive fusion between vertebrae.

What is claimed is:

1. An interspinous distraction implant, characterized in that it includes an O-shaped body, which is oval and laminar.

2. The interspinous distraction implant of claim 1, characterized in that the O-shaped body, which is in lying position, with regard to its lateral ends, has an end wider than the other as well as a thinning at the upper and lower areas.

3. The interspinous distraction implant of claim 2, characterized in that at the bottom of the front part, it includes two rectangular flaps protruding towards the sides with ending edges that serve as anchorage of the implant and avoid the motion thereof outwards of the interspinous space.

4. The interspinous distraction implant of claim 3, characterized in that at the bottom of the front part, it includes a U-shaped extension towards the front, corresponding with the shape of the central body and in the ends of said U there protrude two flaps, the upper flap of which is forward with regard to the lower flap so that both of them are simultaneously stopped by the respective vertebral laminae thus avoiding the rotation of the implant in the transversal axis and positioning its longitudinal axis towards the spinous apophysis.

5. The interspinous distraction implant of claim 4, characterized in that the bottom of the front part, it includes a backwards U-shaped extension, said U being full, in correspondence with the shape of the central body and in the ends of said U there protrude two asymmetric flaps, the upper flap of which is forward with regard to the lower flap so that both of them are simultaneously stopped in the respective vertebral lamina, thus avoiding the rotation of the implant in the transversal axis and positioning its longitudinal axis parallel to the spinous apophysis.

6. The interspinous distraction implant of claim 5, characterized in that in the center of the extension area having a full U shape, there is protuberance lodging a threaded hole to hold a positioning surgical instrument.

7. The interspinous distraction implant of claim 6, characterized in that the material for its constructions is PEEK (Polyetiren-Etiren-ketone).
